(19) United States
(12) Patent Application Publication
(10) Pub. No.: US 2022/0400961 A1
(43) Pub. Date: Dec. 22, 2022

(54) Wrist-Worn Electronic Device, Wrist Size Measurement Method, and Blood Pressure Measurement Method

(71) Applicant: Huawei Technologies Co., Ltd., Shenzhen (CN)

(72) Inventors: Shaojian Wang, Shenzhen (CN); Zhenlong Huang, Shenzhen (CN); Jing Li, Shenzhen (CN); Xiaoyu Fu, Shenzhen (CN)

(21) Appl. No.: 17/784,621

(22) PCT Filed: Dec. 9, 2020
(86) PCT No.: PCT/CN2020/134988

§ 371 Date: Jun. 10, 2022

(30) Foreign Application Priority Data
Dec. 13, 2019 (CN) 201911284010.1

(51) Int. Cl. A61B 5/0205; G04G 21/02; A61B 5/107; A61B 5/00; A44C 5/20

(52) U.S. Cl. CPC A61B 5/0205; G04G 21/025; A61B 5/1075; A61B 5/6843; A61B 5/681; A44C 5/20; A61B 2560/0214; A61B 2562/0247; A61B 5/02108

(57) ABSTRACT

In an embodiment a wrist-worn electronic device includes a main body, a wrist strap connected to the main body, wherein the wrist strap is configured to place the main body on a wrist of a target user, a wrist size determining part configured to measure, by using the wrist strap of the wrist-worn electronic device, a use circumference of the wrist-worn electronic device that matches a wrist size of the target user, and determine the wrist size of the target user based on the use circumference of the wrist-worn electronic device, and a blood pressure determining part configured to detect a pulse wave signal of the target user, measure a measured blood pressure of the target user based on the pulse wave signal, and correct the measured blood pressure of the target user based on the wrist size of the target user thereby obtaining a first corrected blood pressure of the target user.

This application is a National Stage of International Application No. PCT/CN2020/134988, filed on Dec. 9, 2020, which claims priority to Chinese Patent Application No. 201911284010.1, filed on Dec. 13, 2019, both of which are hereby incorporated by reference in their entireties.

### TECHNICAL FIELD

This application relates to the field of wearable device technologies, and in particular, to a wrist-worn electronic device, a wrist size measurement method, and a blood pressure measurement method.

### BACKGROUND

With rapid development of electronic technologies and people's increasing attention to their own health status, wearable devices are increasingly widely used to assist in health assessment.

A blood pressure measurement apparatus is integrated into some wrist-worn electronic devices (for example, a watch or a band) in a conventional technology, and can measure blood pressure of a target user. For example, an airbag and a pressure sensor connected to the airbag are disposed on a watch. The airbag may compress a radial artery of a wrist of the target user in an inflation and deflation process. Therefore, the pressure sensor extracts a pulse wave signal of the target user, and can obtain blood pressure of the target user based on the pulse wave signal.

The wrist-worn electronic device attracts attention due to portability. However, in the conventional technology, a blood pressure measurement result of the wrist-worn electronic device is not accurate enough.

### SUMMARY

The following describes this application from a plurality of aspects. For the following implementations and beneficial effects of the plurality of aspects, refer to each other.

According to a first aspect, an implementation of this application provides a wrist-worn electronic device that can measure blood pressure, including a main body and a wrist strap connected to the main body. The wrist strap is configured to wear the main body on a wrist of a target user. The wrist-worn electronic device further includes: a wrist size determining part, configured to measure, by using the wrist strap of the wrist-worn electronic device, a use circumference of the wrist-worn electronic device that matches a wrist size of the target user, and determine the wrist size of the target user based on the use circumference of the wrist-worn electronic device; and a blood pressure determining part, configured to detect a pulse wave signal of the target user, measure measured blood pressure of the target user based on the pulse wave signal, and correct the measured blood pressure of the target user based on the wrist size of the target user, to obtain first corrected blood pressure of the target user.

According to an implementation of this application, the wrist-worn electronic device can provide the target user with blood pressure corrected based on the wrist size of the target user. This can improve accuracy of blood pressure measurement.

In some implementations, when the wrist size of the target user is greater than a first threshold, the blood pressure determining part subtracts a first specified value from the measured blood pressure to obtain the first corrected blood pressure; and when the wrist size of the target user is less than a second threshold, the blood pressure determining part adds a second specified value to the measured blood pressure to obtain the first corrected blood pressure.

In some implementations, the wrist size of the target user is a wrist circumference of the target user, an optional range of the first threshold is 165 mm to 190 mm, and an optional range of the second threshold is 125 mm to 150 mm.

In some implementations, an optional range of the first specified value is 12 mmHg to 25 mmHg, and an optional range of the second specified value is 1 mmHg to 5 mmHg.

In some implementations, the wrist strap includes a first wrist strap and a second wrist strap that are connected to opposite ends of the main body of the wrist-worn electronic device, a first buckle part is disposed on the first wrist strap, a plurality of second buckle parts that can be fastened to the first buckle part are disposed on the second wrist strap, and the use circumference of the wrist-worn electronic device can be adjusted by fastening different second buckle parts in the plurality of second buckle parts to the first buckle part. The wrist size determining part can detect a second buckle part fastened to the first buckle part in the plurality of second buckle parts, to determine the use circumference of the wrist-worn electronic device.

According to an implementation of this application, measurement of the use circumference of the wrist-worn electronic device is converted into measurement of the second buckle part fastened to the first buckle part. This simplifies a process of measuring the use circumference of the wrist-worn electronic device.

In some implementations, the wrist-worn electronic device includes a power supply and a plurality of detection resistors disposed on the second wrist strap. At least one detection resistor is disposed between adjacent second buckle parts. When one of the plurality of second buckle parts is fastened to the first buckle part, a detection resistor between the second buckle part and the main body can form a closed loop with the power supply. The wrist size determining part can determine, based on a physical parameter corresponding to a resistance of the detection resistor in the closed loop, the second buckle part fastened to the first buckle part in the plurality of second buckle parts.

In some implementations, the wrist-worn electronic device further includes a pressure sensor disposed on an inner circumferential surface of the wrist-worn electronic device. The pressure sensor is configured to measure pressure between the wrist-worn electronic device and the wrist of the target user when the wrist-worn electronic device is worn on the wrist of the target user. The wrist size determining part determines the wrist size of the target user based on the use circumference of the wrist-worn electronic device and the pressure measured by the pressure sensor.

According to an implementation of this application, when determining the wrist size of the target user, the wrist size determining part compensates for a measurement deviation caused by wearing tightness. In this way, when wearing the wrist-worn electronic device, the user can adjust the use circumference of the wrist-worn electronic device based on a personal preference to comfortably wear the wrist-worn electronic device, and does not need to meet a specific wearing requirement. This improves user experience.

In some implementations, the wrist-worn electronic device further includes a pressure sensor disposed on an inner circumferential surface of the wrist-worn electronic device. The pressure sensor is configured to measure pressure between the wrist-worn electronic device and the wrist of the target user when the wrist-worn electronic device is worn on the wrist of the target user. The blood pressure determining part can correct the measured blood pressure of the target user based on the pressure measured by the pressure sensor and the wrist size of the target user, to obtain second corrected blood pressure of the target user.

According to an implementation of this application, in addition to correcting the measured blood pressure based on the wrist size of the target user, the blood pressure determining part further corrects the measured blood pressure based on the pressure measured by the pressure sensor. This further improves accuracy of blood pressure measurement.

According to a second aspect, an implementation of this application provides a wrist-worn electronic device, including a main body and a wrist strap connected to the main body. The wrist strap is configured to wear the main body on a wrist of a target user, and the wrist-worn electronic device further includes: a wrist size determining part, configured to measure, by using the wrist strap of the wrist-worn electronic device, a use circumference of the wrist-worn electronic device that matches a wrist size of the target user, and determine the wrist size of the target user based on the use circumference of the wrist-worn electronic device.

According to an implementation of this application, the wrist size determining part can measure the wrist size of the target user, so that the wrist-worn electronic device can correct measured blood pressure based on the wrist size of the user. This improves accuracy of blood pressure measurement.

In some implementations, the wrist strap includes a first wrist strap and a second wrist strap that are connected to opposite ends of the main body of the wrist-worn electronic device, a first buckle part is disposed on the first wrist strap, a plurality of second buckle parts that can be fastened to the first buckle part are disposed on the second wrist strap, and the use circumference of the wrist-worn electronic device can be adjusted by fastening different second buckle parts in the plurality of second buckle parts to the first buckle part. The wrist size determining part can detect a second buckle part fastened to the first buckle part in the plurality of second buckle parts, to determine the use circumference of the wrist-worn electronic device.

According to an implementation of this application, measurement of the use circumference of the wrist-worn electronic device is converted into measurement of the second buckle part fastened to the first buckle part. This simplifies a process of measuring the use circumference of the wrist-worn electronic device.

In some implementations, the wrist-worn electronic device includes a power supply and a plurality of detection resistors disposed on the second wrist strap. At least one detection resistor is disposed between adjacent second buckle parts. When one of the plurality of second buckle parts is fastened to the first buckle part, a detection resistor between the second buckle part and the main body can form a closed loop with the power supply. The wrist size determining part can determine, based on a physical parameter corresponding to a resistance of the detection resistor in the closed loop, the second buckle part fastened to the first buckle part in the plurality of second buckle parts.

In some implementations, the wrist size determining part further includes a pressure sensor disposed on an inner circumferential surface of the wrist-worn electronic device. The pressure sensor is configured to measure pressure between the wrist-worn electronic device and the wrist of the target user when the wrist-worn electronic device is worn on the wrist of the target user. The wrist size determining part determines the wrist size of the target user based on the use circumference of the wrist-worn electronic device and the pressure measured by the pressure sensor.

According to an implementation of this application, when determining the wrist size of the target user, the wrist size determining part compensates for a measurement deviation caused by wearing tightness. In this way, when wearing the wrist-worn electronic device, the user can adjust the use circumference of the wrist-worn electronic device based on a personal preference to comfortably wear the wrist-worn electronic device, and does not need to meet a specific wearing requirement. This improves user experience.

According to a third aspect, an implementation of this application provides a blood pressure measurement method, performed by a wrist-worn electronic device. The wrist-worn electronic device includes a main body and a wrist strap connected to the main body. The wrist strap is configured to wear the main body on a wrist of a target user. The method includes: measuring, by using the wrist strap of the wrist-worn electronic device, a use circumference of the wrist-worn electronic device that matches a wrist size of the target user, and determining the wrist size of the target user based on the use circumference of the wrist-worn electronic device; detecting a pulse wave signal of the target user, and determining measured blood pressure of the target user based on the pulse wave signal; and correcting the measured blood pressure of the target user based on the wrist size of the target user, to obtain first corrected blood pressure of the target user.

According to an implementation of this application, the wrist-worn electronic device can provide the target user with blood pressure corrected based on the wrist size of the target user. This can improve accuracy of blood pressure measurement.

In some implementations, the correcting the measured blood pressure of the target user based on the wrist size of the target user, to obtain first corrected blood pressure of the target user includes: when the wrist size of the target user is greater than a first threshold, subtracting the first specified value from the measured blood pressure to obtain the first corrected blood pressure; or when the wrist size of the target user is less than a second threshold, adding the second specified value to the measured blood pressure to obtain the first corrected blood pressure.

In some implementations, the wrist size of the target user is a wrist circumference of the target user, an optional range of the first threshold is 165 mm to 190 mm, and an optional range of the second threshold is 125 mm to 150 mm.

In some implementations, an optional range of the first specified value is 12 mmHg to 25 mmHg, and an optional range of the second specified value is 1 mmHg to 5 mmHg.

In some implementations, the wrist strap includes a first wrist strap and a second wrist strap that are connected to opposite ends of the main body of the wrist-worn electronic device, a first buckle part is disposed on the first wrist strap, a plurality of second buckle parts that can be fastened to the first buckle part are disposed on the second wrist strap, and the use circumference of the wrist-worn electronic device can be adjusted by fastening different second buckle parts in the plurality of second buckle parts to the first buckle part; and the measuring a use circumference of the wrist-worn electronic device that matches a wrist size of the target user, and determining the wrist size of the target user based on the use circumference of the wrist-worn electronic device includes: detecting a second buckle part fastened to the first buckle part in the plurality of second buckle parts, to determine the use circumference of the wrist-worn electronic device.

According to an implementation of this application, measurement of the use circumference of the wrist-worn electronic device is converted into measurement of the second buckle part fastened to the first buckle part. This simplifies a process of measuring the use circumference of the wrist-worn electronic device.

In some implementations, the wrist-worn electronic device includes a power supply and a plurality of detection resistors disposed on the second wrist strap. At least one detection resistor is disposed between adjacent second buckle parts. When one of the plurality of second buckle parts is fastened to the first buckle part, a detection resistor between the second buckle part and the main body can form a closed loop with the power supply; and the detecting a second buckle part fastened to the first buckle part in the plurality of second buckle parts, to determine the use circumference of the wrist-worn electronic device includes: determining, based on a physical parameter corresponding to a resistance of the detection resistor in the closed loop, the second buckle part fastened to the first buckle part in the plurality of second buckle parts.

In some implementations, the wrist-worn electronic device further includes a pressure sensor disposed on an inner circumferential surface of the wrist-worn electronic device. The pressure sensor is configured to measure pressure between the wrist-worn electronic device and the wrist of the target user when the wrist-worn electronic device is worn on the wrist of the target user; and the measuring a use circumference of the wrist-worn electronic device that matches a wrist size of the target user, and determining the wrist size of the target user based on the use circumference of the wrist-worn electronic device includes: determining the wrist size of the target user based on the use circumference of the wrist-worn electronic device and the pressure measured by the pressure sensor.

According to an implementation of this application, when determining the wrist size of the target user, a measurement deviation caused by wearing tightness is compensated. In this way, when wearing the wrist-worn electronic device, the user can adjust the use circumference of the wrist-worn electronic device based on a personal preference to comfortably wear the wrist-worn electronic device, and does not need to meet a specific wearing requirement. This improves user experience.

In some implementations, the wrist-worn electronic device further includes a pressure sensor disposed on an inner circumferential surface of the wrist-worn electronic device. The pressure sensor is configured to measure pressure between the wrist-worn electronic device and the wrist of the target user when the wrist-worn electronic device is worn on the wrist of the target user; and the correcting the measured blood pressure of the target user based on the wrist size of the target user, to obtain first corrected blood pressure of the target user includes: correcting the measured blood pressure of the target user based on the pressure measured by the pressure sensor and the wrist size of the target user, to obtain second corrected blood pressure of the target user.

According to an implementation of this application, the measured blood pressure is corrected based on the wrist size of the target user, and the measured blood pressure is further corrected based on the pressure measured by the pressure sensor. This further improves accuracy of blood pressure measurement.

According to a fourth aspect, an implementation of this application provides a wrist size measurement method, performed a wrist-worn electronic device. The wrist-worn electronic device includes a main body and a wrist strap connected to the main body. The wrist strap is configured to wear the main body on a wrist of a target user. The method further includes: measuring, by using the wrist strap of the wrist-worn electronic device, a use circumference of the wrist-worn electronic device that matches a wrist size of the target user, and determining the wrist size of the target user based on the use circumference of the wrist-worn electronic device.

According to an implementation of this application, the wrist size of the target user can be measured, so that the wrist-worn electronic device can correct measured blood pressure based on the wrist size of the user. This improves accuracy of blood pressure measurement.

In some implementations, the wrist strap includes a first wrist strap and a second wrist strap that are connected to opposite ends of the main body of the wrist-worn electronic device, a first buckle part is disposed on the first wrist strap, a plurality of second buckle parts that can be fastened to the first buckle part are disposed on the second wrist strap, and a use circumference of the wrist strap can be adjusted by fastening different second buckle parts in the plurality of second buckle parts to the first buckle part; and the measuring a use circumference of the wrist-worn electronic device that matches a wrist size of the target user, and determining the wrist size of the target user based on the use circumference of the wrist-worn electronic device includes: detecting a second buckle part fastened to the first buckle part in the plurality of second buckle parts, to determine the use circumference of the wrist-worn electronic device.

According to an implementation of this application, measurement of the use circumference of the wrist-worn electronic device is converted into measurement of the second buckle part fastened to the first buckle part. This simplifies a process of measuring the use circumference of the wrist-worn electronic device.

In some implementations, the wrist-worn electronic device includes a power supply and a plurality of detection resistors disposed on the second wrist strap. At least one detection resistor is disposed between adjacent second buckle parts. When one of the plurality of second buckle parts is fastened to the first buckle part, a detection resistor between the second buckle part and the main body can form a closed loop with the power supply; and the detecting a second buckle part fastened to the first buckle part in the plurality of second buckle parts, to determine the use circumference of the wrist-worn electronic device includes: determining, based on a physical parameter corresponding to a resistance of the detection resistor in the closed loop, the second buckle part fastened to the first buckle part in the plurality of second buckle parts.

In some implementations, the wrist-worn electronic device further includes a pressure sensor disposed on an inner circumferential surface of the wrist-worn electronic device. The pressure sensor is configured to measure pressure between the wrist-worn electronic device and the wrist of the target user when the wrist-worn electronic device is worn on the wrist of the target user; and the measuring a use circumference of the wrist-worn electronic device that matches a wrist size of the target user, and determining the wrist size of the target user based on the use circumference of the wrist-worn electronic device includes: determining the wrist size of the target user based on the use circumference of the wrist-worn electronic device and the pressure measured by the pressure sensor.

According to an implementation of this application, when determining the wrist size of the target user, a measurement deviation caused by wearing tightness is compensated. In this way, when wearing the wrist-worn electronic device, the user can adjust the use circumference of the wrist-worn electronic device based on a personal preference to comfortably wear the wrist-worn electronic device, and does not need to meet a specific wearing requirement. This improves user experience.

### BRIEF DESCRIPTION OF THE DRAWINGS

FIG. 1 is a schematic diagram of a structure of a watch according to an embodiment of this application;

FIG. 2 shows a front view of a watch according to an embodiment of this application;

FIG. 3 shows a side view of a watch according to an embodiment of this application;

FIG. 4 shows a rear view of a watch according to an embodiment of this application;

FIG. 5 is a diagram of a use status of a watch according to an embodiment of this application;

FIG. 6 shows an example of a relationship between a wrist size of a user and a deviation value of measured blood pressure;

FIG. 7 shows an example of a relationship between wearing tightness of a watch and a deviation value of measured blood pressure;

FIG. 8 shows a wrist size measurement method according to an embodiment of this application; and

FIG. 9 shows a blood pressure measurement method according to an embodiment of this application.

### DETAILED DESCRIPTION OF ILLUSTRATIVE EMBODIMENTS

The following describes implementations of this application by using specific embodiments. A person skilled in the art may easily learn of other advantages and effects of this application based on content disclosed in this specification. Although this application is described with reference to an example embodiment, it does not mean that a characteristic of this application is limited only to this implementation. On the contrary, a purpose of describing this application with reference to an implementation is to cover another option or modification that may be derived based on claims of this application. To provide an in-depth understanding of this application, the following descriptions include a plurality of specific details. This application may be alternatively implemented without using these details. In addition, to avoid confusion or blurring the focus of this application, some specific details will be omitted from the description. It should be noted that, when there is no conflict, embodiments in this application and the features in embodiments may be mutually combined.

It should be noted that, in this specification, reference numerals and letters in the following accompanying drawings represent similar items. Therefore, once an item is defined in an accompanying drawing, the item does not need to be further defined or interpreted in subsequent accompanying drawings.

In the description of the embodiments, it should be noted that, orientations or position relationships indicated by the terms “on”, “in”, and the like are orientations or position relationships shown based on the accompanying drawings, or orientations or position relationships that are commonly placed when the product in this application is used, and are merely intended to facilitate description of this application and simplify description, instead of indicating or implying that an indicated apparatus or element needs to have a specific orientation and be constructed and operated in a specific orientation, and therefore cannot be understood as limitations on this application.

The terms “first”, “second”, and the like are merely used for distinction and description, and shall not be understood as an indication or implication of relative importance.

In the descriptions of the embodiments, it should be further noted that, unless otherwise specified and limited, the terms “dispose” and “connect” should be understood in a broad sense. For example, a “connection” may be a fixed connection, a detachable connection, or an integrated connection; may be a mechanical connection or an electrical connection; may be a direct connection, or may be an indirect connection through an intermediate medium, or may be an internal connection between two components. The specific meanings about the foregoing terms in the embodiments may be understood by a person of ordinary skill in the art based on specific circumstances.

A wrist-worn electronic device that provides a blood pressure measurement function can detect a pulse wave signal at a wrist of a person, and detect blood pressure based on the pulse wave signal. Different wrist sizes have different impact on the blood pressure measured by the watch. To be specific, for a user with a large wrist size, measured blood pressure of the wrist-worn electronic device is greater than actual blood pressure of the user due to much fat on a wrist. For a user with a small wrist size, measured blood pressure of the wrist-worn electronic device is less than actual blood pressure of the user due to little fat on a wrist.

Based on the foregoing findings, an embodiment of this application provides a wrist-worn electronic device that can measure blood pressure, so that blood pressure measured based on a pulse wave can be corrected based on a wrist size of a target user. In this specification, the “target user” is a current user wearing the watch. The wrist-worn electronic device may be a watch, a band, or another electronic device that is worn on a wrist in a manner of wearing a watch. The following describes the technical solutions of this application by using a watch as an example.

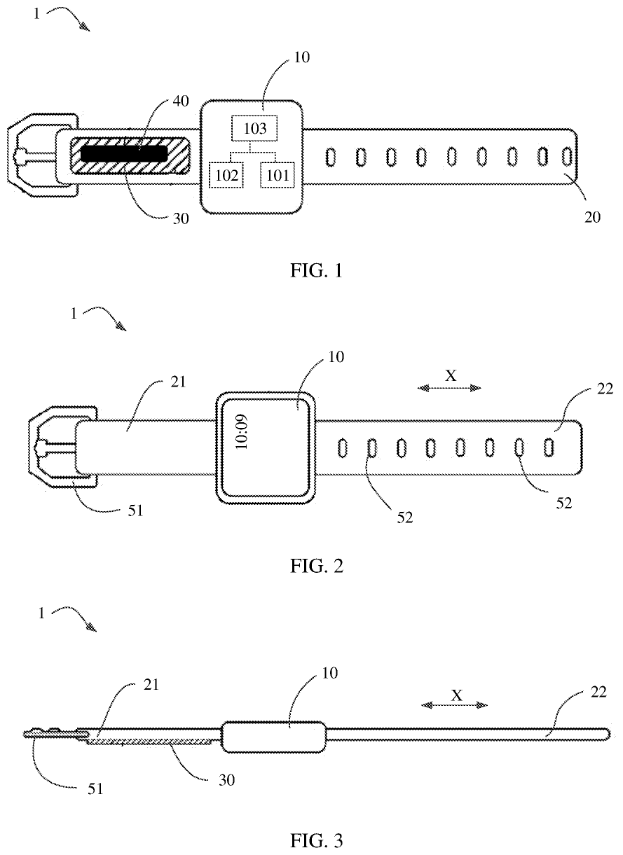

Specifically, as shown in FIG. 1, a watch 1 includes a watch body 10, a watch strap 20, and an airbag 30 and a pressure sensor 40 that are disposed on the watch strap 20. The pressure sensor 40 is configured to measure pressure between the watch 1 and a wrist of a target user. A wrist size determining part 101, a pressure measurement part 102, and a blood pressure determining part 103 are disposed inside the watch body 10. The wrist size determining part 101 can measure parameters from the watch strap 20 that are used to indicate a wrist size (for example, a wrist circumference, and a wrist diameter) of the target user, and determine the wrist size of the user based on the parameters. The blood pressure determining part 103 includes a pulse wave signal detection unit, determines blood pressure of the target user based on a detected pulse wave signal, and can correct measured blood pressure based on the wrist size of the target user to obtain corrected blood pressure. Optionally, the pressure measurement part 102 is configured to collect a pressure from the pressure sensor 40, and output the pressure to the blood pressure determining part 103, to further correct the blood pressure that is corrected based on the wrist size. The watch 1 in this embodiment can provide the target user with blood pressure corrected based on the wrist size of the target user and the pressure measured by the pressure sensor 40. This can improve accuracy of blood pressure measurement.

When the watch 1 is used, the target user may adjust, by adjusting an effective use length of the watch strap, a circumference of the watch 1 to match the wrist size of the target user, so that the watch 1 is worn comfortably. In this specification, the circumference of the watch 1 that matches the wrist size of the target user is referred to as “a use circumference of the watch”. In this embodiment, the wrist size determining part 101 is configured to measure the use circumference of the watch by using the watch strap, and determine the wrist size of the target user based on the use circumference of the watch. For example, the wrist size determining part 101 may determine a wrist circumference of the target user as a value that is the same as the use circumference of the watch. In addition, the wrist size determining part 101 may be software, hardware, or a combination of software and hardware.

Specifically, refer to FIG. 1 to FIG. 5. The watch 1 according to implementations of this application includes the watch body 10 and the watch strap 20. The watch strap 20 includes a first watch strap 21 and a second watch strap 22 that are connected to two opposite ends of the watch body 10. A first buckle part 51 is disposed on the first watch strap 21, and a plurality of second buckle parts 52 that can be fastened to the first buckle part 51 are disposed on the second watch strap 22. Different second buckle parts 52 in the plurality of second buckle parts 52 are fastened to the first buckle part 51, to adjust an effective use length of the watch strap, and further adjust the use circumference of the watch.

Forms of the first buckle part 51 and the second buckle part 52 are not limited in this embodiment. For example, the first buckle part 51 and the second buckle part 52 may be buckle parts that can be fastened to each other in a folding buckle, a pin buckle, a press buckle, a butterfly buckle, a hook, or the like. Optionally, the plurality of second buckle parts 52 are distributed at equal intervals along a length direction (an X direction shown in FIG. 2 to FIG. 5) of the second watch strap 22.

When the watch 1 is being worn, the wrist size determining part 101 can detect a second buckle part 52 fastened to the first buckle part 51 in the plurality of second buckle parts 52 (for ease of description, the second buckle part 52 fastened to the first buckle part 51 in the plurality of second buckle parts 52 is referred to as a “target second buckle part P” for short), to determine the use circumference of the watch. For example, refer to FIG. 5. A length d1 of an effective use section (a part that is of the second watch strap 22 and that is located between the watch body 10 and the target second buckle part P) of the second watch strap 22 is obtained by detecting a location of the target second buckle part P, and then the use circumference of the watch is obtained by accumulating the length d1 of the effective use section of the second watch strap 22, a length d2 of the watch body 10, a length d3 of the first watch strap 21, and the like. In this embodiment, measurement of the use circumference of the watch is converted into measurement of the target second buckle part P. This simplifies a measurement process of the use circumference of the watch 1.

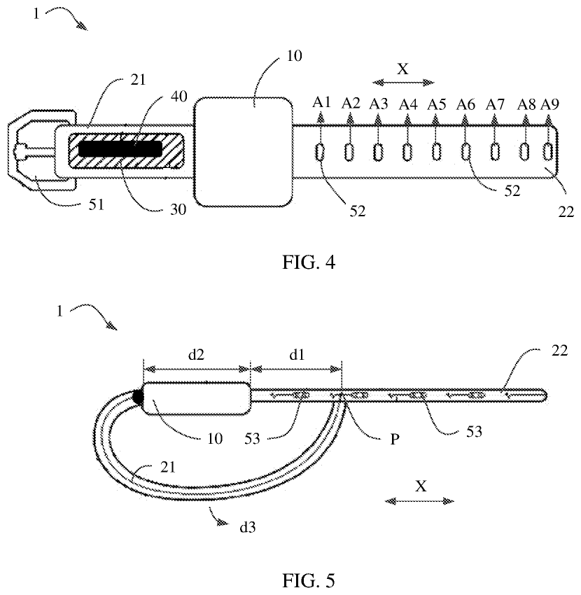

Based on the technical solution provided in this embodiment, Table 1 shows an example of a correspondence between the location of the target second buckle part P and the use circumference of the watch. For locations represented by A1 to A9, refer to FIG. 4.

TABLE 1

| Location of the target | Use circumference of |
|---|---|
| second buckle part | the watch (mm) |
| A1 | 137 |
| A2 | 144 |
| A3 | 152 |
| A4 | 161 |
| A5 | 170 |
| A6 | 180 |
| A7 | 190 |
| A8 | 200 |
| A9 | 211 |

Refer to FIG. 5. In this embodiment, the watch 1 includes a power supply (not shown) and a plurality of detection resistors 53 disposed on the second watch strap 22. When the first buckle part 51 is fastened to the second buckle part 52, a detection resistor 53 on the effective use section of the second watch strap 22 and the power supply may jointly form a closed length detection loop. To be specific, in this embodiment, the first buckle part 51 and the second buckle part 52 form a switch of the length detection loop. When the first buckle part 51 is fastened to the second buckle part 52, the length detection loop is closed. When the first buckle part 51 and the second buckle part 52 are detached, the length detection loop is open. Based on this, an implementation form of the switch is not limited in this application. For example, a material of the first buckle part 51 and a material of the second buckle part 52 are both conductive materials, and when the first buckle part 51 is fastened to the second buckle part 52, the length detection loop can be closed. Alternatively, a conductive sheet is disposed on each of the first buckle part 51 and the second buckle part 52, and when the first buckle part 51 is fastened to the second buckle part 52, the conductive sheet on the first buckle part 51 contacts the conductive sheet on the second buckle part 52, and therefore, the length detection loop is closed.

A form of the power supply is not limited in this embodiment. For example, the power supply may be a lithium battery, a power supply chip, or the like, provided that the power supply can supply power to the detection resistor 53. A location at which the power supply is disposed is not limited in this embodiment. The power supply may be disposed on the watch body 10, the first watch strap 21, or the second watch strap 22, provided that the power supply can form a closed loop with the detection resistor 53.

At least one detection resistor 53 is disposed between adjacent second buckle parts 52. Therefore, when different second buckle parts 52 are fastened to the first buckle part 51, a detection resistor 53 (briefly referred to as an “effective resistor”) on the effective use section of the second watch strap 22 has different resistances (the resistance is a total resistance of the foregoing effective resistors, and is briefly referred to as an “effective resistance”). Optionally, the detection resistors 53 between the adjacent second buckle parts 52 have a same resistance. Optionally, the detection resistors 53 are connected in series with each other.

In this embodiment, the wrist size determining part 101 can determine a location of the target second buckle part P based on a physical parameter corresponding to a resistance of an effective resistor, to determine the use circumference of the watch 1. To be specific, in this embodiment, the physical parameter is used as a parameter indicating the use circumference of the watch, and the use circumference of the watch is obtained based on the physical parameter. A type of the physical parameter is not limited in this application. A person skilled in the art may determine a specific physical parameter based on a type of the length detection loop, for example, divided voltages at two ends of the effective resistor, a current passing through the effective resistor, and an effective resistance, provided that the physical parameter can correspond to the effective resistance.

The wrist size determining part 101 may further determine the location of the target second buckle part P in another manner. For example, in another embodiment, a pressure sensor is disposed on each second buckle part 52. It can be understood that, compared with the second buckle part 52 that is not fastened to the first buckle part 51, a pressure sensor on the target second buckle part P may detect larger pressure. Therefore, the wrist size determining part 101 can determine the location of the target second buckle part P based on pressure detected by the pressure sensor on each second buckle part 52.

In still another embodiment, a detection chip configured to detect the location of the target second buckle part P is disposed in the watch body 10. The detection chip may be a dedicated chip configured to detect the location of the target second buckle part P, or may be a part of the wrist size determining part 101 in the watch 1. The first buckle part 51 is connected to a power supply pin of the detection chip, and each second buckle part 52 is connected to a different detection pin of the detection chip. When the first buckle part 51 is fastened to the second buckle part 52, the first buckle part 51 is electrically connected to the second buckle part 52. It can be understood that, when the watch 1 is being worn, a detection pin connected to the target second buckle part P has a high level, and another detection pin has a low level. Therefore, the wrist size determining part 101 determines the location of the target second buckle part P by obtaining a level status of each detection pin.

When the target user wears the watch 1, the watch 1 is adjusted to different tightness based on different personal preferences. Specifically, if the target user prefers to adjust the watch 1 to a tight state, the actual wrist size of the target user may be greater than the wrist size determined based on the use circumference of the watch. If the target user prefers to adjust the watch 1 to a loose state, the actual wrist size of the target user may be less than the wrist size determined based on the use circumference of the watch.

To reduce impact of an improper wearing manner on a measurement result, the pressure sensor 40 is disposed on an inner circumferential surface of the watch 1 provided in this embodiment. The pressure sensor 40 is configured to measure pressure between the watch 1 and the wrist of the target user, to indicate wearing tightness of the watch 1. Herein, the inner circumferential surface of the watch 1 is a surface that faces the wrist of the target user when the watch 1 is being worn. In this embodiment, the wrist size determining part 101 can determine the wrist size of the target user based on the use circumference of the watch and the pressure measured by the pressure sensor 40. To be specific, in an embodiment of this application, when determining the wrist size of the target user, the wrist size determining part 101 compensates for a measurement deviation caused by wearing tightness. In this way, when wearing the watch 1, the user can adjust the use circumference of the watch based on a personal preference to comfortably wear the watch, and does not need to meet a specific wearing requirement. This improves user experience.

Specifically, when the pressure measured by the pressure sensor 40 is large, the wrist size of the target user that is determined based on the use circumference of the watch may be increased accordingly, to obtain a final wrist size of the target user. When the pressure measured by the pressure sensor 40 is small, the wrist size of the target user that is determined based on the use circumference of the watch may be decreased accordingly, to obtain a final wrist size of the target user.

According to the technical solution provided in this embodiment, Table 2 shows an example of a correspondence between pressure measured by the pressure sensor 40 and wearing tightness of the watch 1. Specific values of P1 to P4 may be measured through an experiment with reference to wearing experience of the watch 1. The wrist size determining part 101 may add a wrist circumference adjustment value listed in Table 2 on the wrist circumference of the target user that is obtained based on the use circumference of the watch, to obtain the final wrist circumference of the target user.

For example, based on the examples provided in Table 1 and Table 2, if a location of the target second buckle part P is A4, and the pressure measured by the pressure sensor 40 is from P3 to P4, the wrist circumference of the target user determined by the wrist size determining part 101 may be 161 mm+2 mm, that is, 163 mm.

TABLE 2

| | Pressure measured by the pressure sensor | | | | |
|---|---|---|---|---|---|
| | <P1 | P1 to P2 | P2 to P3 | P3 to P4 | >P4 |
| Wearing tightness | Loose | Moderately | Moderate | Moderately | Tight |
| | | loose | | tight | |
| Wrist circumference | −4 to −6 | −1 to −3 | 0 | 1 to 3 | 4 to 6 |
| adjustment value (mm) | | | | | |

Vibration of blood pressure, a blood flow velocity, a blood flow, and transmission of the deformation and vibration of the vascular wall in the vascular system as intermittent contraction and relaxation of the heart are collectively referred to as pulse waves. As described above, the blood pressure determining part 103 in this embodiment can obtain measured blood pressure of the target user based on the pulse wave signal of the target user. Based on this, a specific blood pressure measurement method is not limited in this application. In this embodiment, the blood pressure measurement method is an oscillographic method. Refer to FIG. 3 and FIG. 4. The airbag 30 and a pressure measurement apparatus (not shown) connected to the airbag 30 are disposed on the watch 1. The airbag 30 compresses a radial artery of the wrist of the target user in an inflation and deflation process. Therefore, the pressure measurement apparatus obtains the pulse wave signal of the target user, and the blood pressure determining part 103 can determine the measured blood pressure of the target user based on the pulse wave signal. In other embodiments, the blood pressure measurement method may be a photoplethysmography method, a volume clamp method, or the like.

As described above, in this embodiment, the blood pressure determining part 103 can further correct the measured blood pressure of the target user based on the wrist size of the target user, to obtain a first corrected blood pressure of the target user. This improves blood pressure measurement precision.

FIG. 6 shows an example of a relationship between the wrist size of the user and a deviation value (that is, a difference between the measured blood pressure of the target user and the actual blood pressure of the target user) of the measured blood pressure. For a target user with a large wrist size, measured blood pressure of the watch 1 is greater than an actual blood pressure of the user. Therefore, in this embodiment, when the wrist size of the target user is greater than a first threshold, the blood pressure determining part 103 subtracts a first specified value based on the measured blood pressure to obtain a first corrected blood pressure. For a target user with a small wrist size, measured blood pressure of the watch 1 is less than an actual blood pressure of the user. When the wrist size of the target user is less than a second threshold, the blood pressure determining part 103 adds a second specified value to the measured blood pressure to obtain a first corrected blood pressure.

In this embodiment, the wrist size of the target user is a wrist circumference of the target user. Correspondingly, based on an experiment result, an optional range of the first threshold is 165 mm to 190 mm, for example, 180 mm; and an optional range of the second threshold is 125 mm to 150 mm, for example, 140 mm.

Further, the first specified value and the second specified value may also be determined based on the experiment result. For example, an optional range of the first specified value is 12 mmHg to 25 mmHg, for example, 17 mmHg; and an optional range of the second specified value is 1 mmHg to 5 mmHg, for example, 3 mmHg.

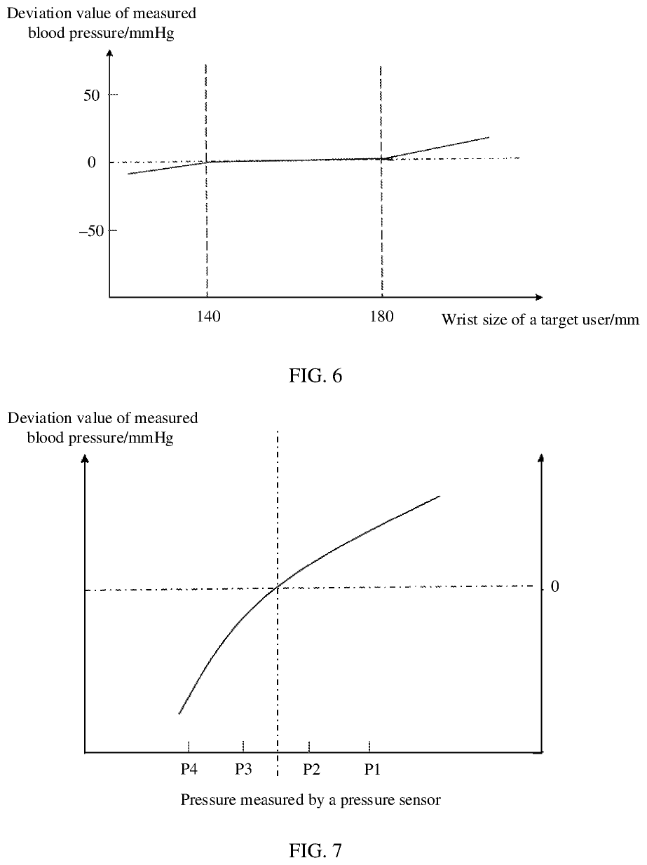

In addition, different wearing tightness of the watch 1 also causes different deviations to the measured blood pressure of the watch 1. FIG. 7 shows an example of a relationship between wearing tightness of the watch 1 and a deviation value (that is, a difference between the measured blood pressure of the target user and the actual blood pressure of the target user) of the measured blood pressure. To be specific, if the watch 1 is worn extremely loosely, there is space between the airbag 30 and the skin, and atmospheric pressure in the airbag 30 is greater than pressure for compressing the skin. Consequently, a pulse wave signal moves towards high pressure, and the measured blood pressure is greater than the actual blood pressure of the user. If the airbag 30 is worn extremely tightly, atmospheric pressure in the airbag 30 is less than pressure for compressing the skin. Consequently, a pulse wave signal moves towards low pressure, and the measured blood pressure is less than the actual blood pressure of the user.

Therefore, in an embodiment of this application, in addition to correcting the measured blood pressure based on the wrist size of the target user, the blood pressure determining part 103 further corrects the measured blood pressure based on the pressure measured by the pressure sensor 40, to obtain second corrected blood pressure. This further improves accuracy of blood pressure measurement. For example, based on the pressure measured by the pressure sensor 40, a blood pressure adjustment value in Table 3 may be added to the first corrected blood pressure to obtain the second corrected blood pressure. A range of the blood pressure adjustment value may be determined based on the experiment. For example, based on the example provided in Table 3, when the pressure measured by the pressure sensor 40 is from P3 to P4, 10 mmHg may be added to the first corrected blood pressure to obtain the second corrected blood pressure.

TABLE 3

| | Pressure measured by the pressure sensor | | | | |
|---|---|---|---|---|---|
| | <P1 | P1 to P2 | P2 to P3 | P3 to P4 | >P4 |
| Wearing tightness | Loose | Moderately | Moderate | Moderately | Tight |
| | | loose | | tight | |
| Blood pressure | −15 to −20 | −10 to −15 | 0 | 8 to 12 | 12 to 18 |
| adjustment value (mmHg) | | | | | |

The following describes a wrist size measurement method and a blood pressure measurement method according to some embodiments. The methods can be implemented in the watch 1 described above in this specification. For content that is not described in the foregoing embodiments of watch 1, refer to the following method embodiments. Similarly, for content that is not described in the method embodiments, refer to the foregoing embodiments of watch 1.

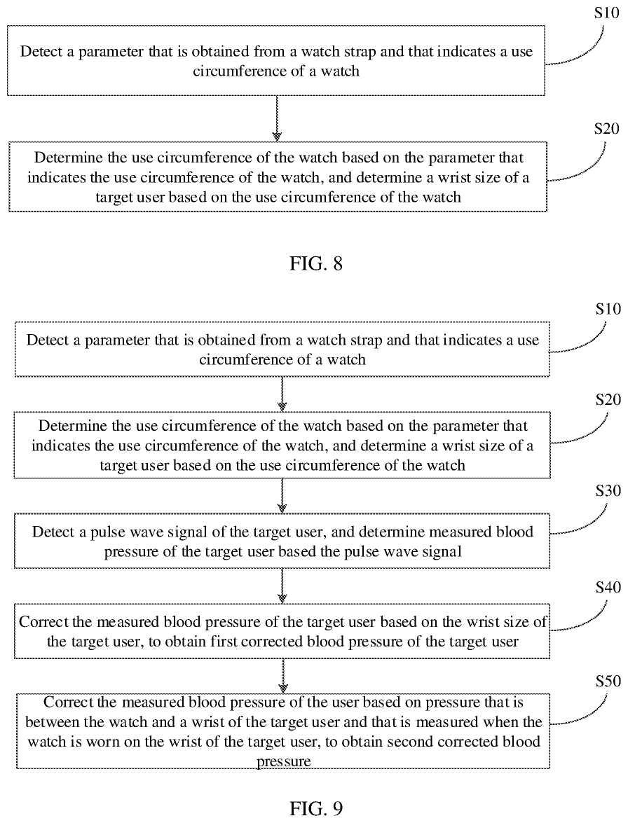

Refer to FIG. 8 and FIG. 1 to FIG. 5. An embodiment of this application provides a method for measuring a wrist size of a target user. The method is performed by the watch 1 provided in embodiments of this application, and specifically includes the following steps.

S10: Detect a parameter that indicates a use circumference of the watch and that is obtained from a watch strap, for example, divided voltages at two ends of an effective resistor (that is, a detection resistor 53 on an effective use section of a second watch strap 22), a current passing through the effective resistor, and an effective resistance.

S20: Determine the use circumference of the watch based on the parameter that indicates the use circumference of the watch, and determine the wrist size of the target user based on the use circumference of the watch.

In some possible implementations, the watch strap 20 includes a first watch strap 21 and the second watch strap 22 that are connected to two opposite ends of a watch body 10 of the watch 1. A first buckle part 51 is disposed on the first watch strap 21, and a plurality of second buckle parts 52 that can be fastened to the first buckle part 51 are disposed on the second watch strap 22. Different second buckle parts 52 in the plurality of second buckle parts 52 are fastened to the first buckle part 51, to adjust an effective use length of the watch strap 20. In step S10, the use circumference of the watch is determined by detecting a second buckle part 52 fastened to the first buckle part 51 in the plurality of second buckle parts 52. Forms of the first buckle part 51 and the second buckle part 52 are not limited in this embodiment. For example, the first buckle part 51 and the second buckle part 52 may be buckle parts that can be fastened to each other in a form of folding buckle, a pin buckle, a press buckle, a butterfly buckle, a hook, or the like.

In some optional implementations, the watch 1 includes a power supply and a plurality of detection resistors 53 disposed on the second watch strap 22. At least one detection resistor 53 is disposed between adjacent second buckle parts 52. When one of the plurality of second buckle parts 52 is fastened to the first buckle part 51, a detection resistor 53 between the second buckle part 52 and the watch body 10 can form a closed loop with the power supply. In step S10, the second buckle part 52 fastened to the first buckle part 51 in the plurality of second buckle parts 52 is determined based on a physical parameter corresponding to a resistance value of the detection resistor 53 in the closed loop.

In some optional implementations, the watch 1 further includes a pressure sensor 40 disposed on an inner circumferential surface of the watch 1. The pressure sensor 40 is configured to measure pressure between the watch 1 and a wrist of the target user when the watch 1 is worn on the wrist of the target user. In step S10, the wrist size of the target user is determined based on the use circumference of the watch and the pressure measured by the pressure sensor 40. When the pressure measured by the pressure sensor 40 is large, the wrist size of the target user that is determined based on the use circumference of the watch may be increased accordingly, to obtain a final wrist size of the target user. When the pressure measured by the pressure sensor 40 is small, the wrist size of the target user that is determined based on the use circumference of the watch may be decreased accordingly, to obtain a final wrist size of the target user.

Refer to FIG. 9 and FIG. 1 to FIG. 5. Another embodiment of this application provides a blood pressure measurement method. The method is performed by the watch 1 provided in embodiments of this application. The blood pressure measurement method provided in this embodiment is based on the method for measuring a wrist size of a target user in the previous embodiment. To be specific, the method in this embodiment includes step S10 and step S20 in the previous embodiment. Based on this, the method in this embodiment further includes the following steps.

S30: Detect a pulse wave signal of the target user, and determine measured blood pressure of the target user based on the pulse wave signal. In this embodiment, the blood pressure measurement method is an oscillographic method, an airbag 30 and a pressure measurement apparatus connected to the airbag 30 are disposed on the watch 1. The airbag 30 compresses a radial artery of a wrist of the target user in an inflation and deflation process. Therefore, the pressure measurement apparatus obtains the pulse wave signal of the target user, and a blood pressure determining part 103 can determine the measured blood pressure of the target user based on the pulse wave signal. In other embodiments, the blood pressure measurement method may be a photoplethysmography method, a volume clamp method, or the like.

S40: Correct the measured blood pressure of the target user based on the wrist size of the target user, to obtain a first corrected blood pressure of the target user.

In some implementations, in step S40, when the wrist size of the target user is greater than a first threshold, the blood pressure determining part 103 subtracts a first specified value from the measured blood pressure to obtain the first corrected blood pressure; and when the wrist size of the target user is less than a second threshold, the blood pressure determining part 103 adds a second specified value to the measured blood pressure to obtain the first corrected blood pressure.

In some optional implementations, in step S30, the wrist size of the target user is a wrist circumference of the target user. An optional range of the first threshold is 165 mm to 190 mm, for example, 180 mm; and an optional range of the second threshold is 125 mm to 150 mm, for example, 140 mm.

In some optional implementations, in step S30, an optional range of the first specified value is 12 to 25 mmHg, for example, 17 mmHg; and an optional range of the second specified value is 1 to 5 mmHg, for example, 3 mmHg.

S50: Correct the measured blood pressure of the user based on pressure that is between the watch and the wrist of the target user and that is measured when the watch is worn on the wrist of the target user, to obtain second corrected blood pressure.

The watch 1 further includes a pressure sensor 40 disposed on an inner circumferential surface of the watch 1. The pressure sensor 40 is configured to measure the pressure between the watch 1 and the wrist of the target user when the watch 1 is worn on the wrist of the target user. As further correction based on a result obtained in step S40, in step S50, the measured blood pressure of the target user is corrected based on the pressure measured by the pressure sensor 40 and the wrist size of the target user, to obtain the second corrected blood pressure of the target user.

The previous describes the technical solutions of this application by using a watch as an example. Based on the foregoing described specific implementations of this application, a person skilled in the art may understand that the structure and the method described in this application are also applicable to a band or another electronic device that is worn on a wrist in a manner of wearing a watch.

In conclusion, the foregoing embodiments in this application are merely illustrative of principles and effects of this application, but are not intended to limit this application. Any person skilled in the art may modify or alter the above embodiments without departing from the spirit and scope of this application. Therefore, all equivalent modifications or alterations made by persons having ordinary knowledge in the technical field without departing from the spirit and technical idea disclosed in this application shall still be covered by the claims of this application.

1. A wrist-worn electronic device comprising:
a main body;
a wrist strap connected to the main body, wherein the wrist strap is configured to place the main body on a wrist of a target user;
a wrist size determining part configured to:
measure, by using the wrist strap of the wrist-worn electronic device, a use circumference of the wrist-worn electronic device that matches a wrist size of the target user, and
determine the wrist size of the target user based on the use circumference of the wrist-worn electronic device; and
a blood pressure determining part configured to:
detect a pulse wave signal of the target user,
measure a measured blood pressure of the target user based on the pulse wave signal, and
correct the measured blood pressure of the target user based on the wrist size of the target user thereby obtaining a first corrected blood pressure of the target user.

2. The wrist-worn electronic device according to claim 1,
wherein, when the wrist size of the target user is greater than a first threshold, the blood pressure determining part is configured to subtract a first specified value from the measured blood pressure to obtain the first corrected blood pressure, and
wherein, when the wrist size of the target user is less than a second threshold, the blood pressure determining part is configured to add a second specified value to the measured blood pressure thereby obtaining the first corrected blood pressure.

3. The wrist-worn electronic device according to claim 1,
wherein the wrist strap comprises a first wrist strap and a second wrist strap that are connected to opposite ends of the main body of the wrist-worn electronic device,
wherein a first buckle part is disposed on the first wrist strap,
wherein a plurality of second buckle parts configured to be fastened to the first buckle part are disposed on the second wrist strap, and
wherein the use circumference of the wrist-worn electronic device is adjustable by fastening different second buckle parts in the plurality of second buckle parts to the first buckle part; and
wherein the wrist size determining part is configured to detect a second buckle part fastened to the first buckle part in the plurality of second buckle parts thereby determining the use circumference of the wrist-worn electronic device.

4. The wrist-worn electronic device according to claim 3, further comprising:
a power supply; and
a plurality of detection resistors disposed on the second wrist strap,
wherein at least one detection resistor is disposed between adjacent second buckle parts,
wherein, when one of the plurality of second buckle parts is fastened to the first buckle part, a detection resistor between the second buckle part and the main body is configured to form a closed loop with the power supply, and
wherein the wrist size determining part is configured to determine, based on a physical parameter corresponding to a resistance of the detection resistor in the closed loop, that the second buckle part is fastened to the first buckle part in the plurality of second buckle parts.

5. The wrist-worn electronic device according to claim 1, further comprising:
a pressure sensor disposed on an inner circumferential surface of the wrist-worn electronic device,
wherein the pressure sensor is configured to measure pressure between the wrist-worn electronic device and the wrist of the target user when the wrist-worn electronic device is worn on the wrist of the target user, and
wherein the wrist size determining part is configured to determine the wrist size of the target user based on the use circumference of the wrist-worn electronic device and the pressure measured by the pressure sensor.

6. The wrist-worn electronic device according to claim 1, further comprising:
a pressure sensor disposed on an inner circumferential surface of the wrist-worn electronic device,
wherein the pressure sensor is configured to measure pressure between the wrist-worn electronic device and the wrist of the target user when the wrist-worn electronic device is worn on the wrist of the target user, and
wherein the blood pressure determining part is configured to correct the measured blood pressure of the target user based on the pressure measured by the pressure sensor and the wrist size of the target user thereby obtaining a second corrected blood pressure of the target user.

7-10. (canceled)

11. A blood pressure measurement method performed by a wrist-worn electronic device, wherein the wrist-worn electronic device comprises a main body and a wrist strap connected to the main body, and wherein the wrist strap is configured to place the main body on a wrist of a target user, the method comprising:
measuring, by the wrist strap of the wrist-worn electronic device, a use circumference of the wrist-worn electronic device that matches a wrist size of the target user;
determining the wrist size of the target user based on the use circumference of the wrist-worn electronic device;
detecting a pulse wave signal of the target user;
determining a measured blood pressure of the target user based on the pulse wave signal; and
correcting the measured blood pressure of the target user based on the wrist size of the target user thereby obtaining a first corrected blood pressure of the target user.

12. The method according to claim 11, wherein correcting the measured blood pressure of the target user based on the wrist size of the target user comprises:
when the wrist size of the target user is greater than a first threshold, subtracting a first specified value from the measured blood pressure thereby obtaining the first corrected blood pressure; and
when the wrist size of the target user is less than a second threshold, adding a second specified value to the measured blood pressure thereby obtaining the first corrected blood pressure.

13. The method according to claim 11,
wherein the wrist strap comprises a first wrist strap and a second wrist strap that are connected to opposite ends of the main body,
wherein a first buckle part is disposed on the first wrist strap,
wherein a plurality of second buckle parts configured to be fastened to the first buckle part are disposed on the second wrist strap,
wherein the use circumference of the wrist-worn electronic device is adjustable by fastening different second buckle parts in the plurality of second buckle parts to the first buckle part,
wherein measuring the use circumference of the wrist-worn electronic device that matches the wrist size of the target user, and determining the wrist size of the target user based on the use circumference of the wrist-worn electronic device comprises detecting a second buckle part fastened to the first buckle part in the plurality of second buckle parts thereby determining the use circumference of the wrist-worn electronic device.

14. The method according to claim 13,
wherein the wrist-worn electronic device comprises a power supply and a plurality of detection resistors disposed on the second wrist strap,
wherein at least one detection resistor is disposed between adjacent second buckle parts,
wherein, when one of the plurality of second buckle parts is fastened to the first buckle part, a detection resistor between the second buckle part and the main body is configured to form a closed loop with the power supply, and
wherein detecting the second buckle part fastened to the first buckle part in the plurality of second buckle parts comprises determining, based on a physical parameter corresponding to a resistance of the detection resistor in the closed loop, the second buckle part fastened to the first buckle part in the plurality of second buckle parts.

15. The method according to claim 11,
wherein the wrist-worn electronic device further comprises a pressure sensor disposed on an inner circumferential surface of the wrist-worn electronic device,
wherein the pressure sensor is configured to measure pressure between the wrist-worn electronic device and the wrist of the target user when the wrist-worn electronic device is worn on the wrist of the target user, and
wherein measuring the use circumference of the wrist-worn electronic device that matches the wrist size of the target user, and determining the wrist size of the target user based on the use circumference of the wrist-worn electronic device comprises determining the wrist size of the target user based on the use circumference of the wrist-worn electronic device and the pressure measured by the pressure sensor.

16. The method according to claim 11,
wherein the wrist-worn electronic device further comprises a pressure sensor disposed on an inner circumferential surface of the wrist-worn electronic device,
wherein the pressure sensor is configured to measure pressure between the wrist-worn electronic device and the wrist of the target user when the wrist-worn electronic device is worn on the wrist of the target user, and
wherein correcting the measured blood pressure of the target user based on the wrist size of the target user comprises correcting the measured blood pressure of the target user based on the pressure measured by the pressure sensor and the wrist size of the target user thereby obtaining a second corrected blood pressure of the target user.

17. A wrist size measurement method, performed by a wrist-worn electronic device, wherein the wrist-worn electronic device comprises a main body and a wrist strap connected to the main body, and wherein the wrist strap is configured to place the main body on a wrist of a target user, the method comprising
measuring, by using the wrist strap of the wrist-worn electronic device, a use circumference of the wrist-worn electronic device that matches a wrist size of the target user; and
determining the wrist size of the target user based on the use circumference of the wrist-worn electronic device.

18. The method according to claim 17,
wherein the wrist strap comprises a first wrist strap and a second wrist strap that are connected to opposite ends of the main body of the wrist-worn electronic device,
wherein a first buckle part is disposed on the first wrist strap,
wherein a plurality of second buckle parts, which is configured to be fastened to the first buckle part, is disposed on the second wrist strap, and an effective use length of the wrist strap is adjustable by fastening different second buckle parts in the plurality of second buckle parts to the first buckle part, and
wherein measuring the use circumference of the wrist-worn electronic device that matches the wrist size of the target user, and determining the wrist size of the target user based on the use circumference of the wrist-worn electronic device comprises detecting a second buckle part fastened to the first buckle part in the plurality of second buckle parts thereby determining the use circumference of the wrist-worn electronic device.

19. The method according to claim 18,
wherein the wrist-worn electronic device comprises a power supply and a plurality of detection resistors disposed on the second wrist strap,
wherein at least one detection resistor is disposed between adjacent second buckle parts,
wherein, when one of the plurality of second buckle parts is fastened to the first buckle part, a detection resistor between the second buckle part and the main body is configured to form a closed loop with the power supply,
wherein detecting the second buckle part fastened to the first buckle part in the plurality of second buckle parts comprises determining, based on a physical parameter corresponding to a resistance of the detection resistor in the closed loop, the second buckle part fastened to the first buckle part in the plurality of second buckle parts.

20. The method according to claim 17,
wherein the wrist-worn electronic device further comprises a pressure sensor disposed on an inner circumferential surface of the wrist-worn electronic device,
wherein the pressure sensor is configured to measure pressure between the wrist-worn electronic device and the wrist of the target user when the wrist-worn electronic device is worn on the wrist of the target user, and
wherein measuring the use circumference of the wrist-worn electronic device that matches the wrist size of the target user, and determining the wrist size of the target user based on the use circumference of the wrist-worn electronic device comprises determining the wrist size of the target user based on the use circumference of the wrist-worn electronic device and the pressure measured by the pressure sensor.

21. The wrist-worn electronic device according to claim 3, wherein a conductive sheet is disposed on each of the first buckle part and the second buckle part.

22. The wrist-worn electronic device according to claim 1, further comprising:
a detection part disposed in the main body,
wherein a first buckle part is connected to the detection part,
wherein each second buckle part is connected to a different pin of the detection part,
wherein, when the first buckle part is connected to a target second buckle part, a level status of a corresponding pin that connects to the target second buckle part is different from a level status of a pin that does not connect to the target second buckle part.

23. The method according to claim 13, wherein a conductive sheet is disposed on each of the first buckle part and the second buckle part.

24. The method according to claim 13,
wherein the electronic device comprises a detection module disposed in the main body,
wherein the first buckle part is connected to the detection module,
wherein each second buckle part is connected to a different pin of the detection module,
wherein the method further comprises, when the first buckle part is connected to a target second buckle part, determining a level status of each pin of the detection module, wherein a level status of a corresponding pin that connects to the target second buckle part is different from a level status of a pin that does not connect to the target second buckle part.
